(19) United States
(12) Patent Application Publication
(10) Pub. No.: US 2003/0028977 A1
(43) Pub. Date: Feb. 13, 2003

(54) Oxidation dyeing composition for keratinous fibres and dyeing method using same

(76) Inventor: Gerard Lang, Saint Prix (FR)

Correspondence Address: FINNEGAN, HENDERSON, FARABOW, GARRETT &, DUNNER LLP, 1300 I STREET, NW, WASHINGTON, DC, 20005, US

(21) Appl. No.: 09/959,702

(22) PCT Filed: Mar. 6, 2001
(86) PCT No.: PCT/FR01/00663

(51) Int. Cl. A61K 7/13

(52) U.S. Cl. 8/405; 8/406; 8/415

(57) ABSTRACT

The invention concerns an oxidation dyeing composition for keratinous fibers, and in particular human keratinous fibers such as hair comprising, in a suitable dyeing medium, at least a first oxidation base selected among certain substituted para-phenylenediamine derivatives and their addition salts with an acid, and at least a second selected oxidation base, and the dyeing method using said composition.

The subject of the invention is a composition for the oxidation dyeing of keratinous fibers, and in particular human keratinous fibers such as hair, comprising, in an appropriate dyeing medium, at least a first oxidation base chosen from certain substituted derivatives of para-phenylenediamine and their addition salts with an acid and at least a second selected oxidation base, and the dyeing method using this composition.

It is known to dye keratinous fibers, and in particular human hair, with dyeing compositions containing oxidation dye precursors, in particular ortho- or para-phenylenediamines, ortho- or para-aminophenols, heterocyclic bases, generally called oxidation bases. Oxidation dye precursors, or oxidation bases, are colorless or faintly colored compounds which, when combined with oxidizing products, can give rise, by a process of oxidative condensation, to colored and coloring compounds.

It is also known that it is possible to vary the shades obtained with these oxidation bases by combining them with couplers or color modifiers, the latter being chosen in particular from aromatic meta-diamines, meta-aminophenols, meta-diphenols and certain heterocyclic compounds.

The variety of molecules used in oxidation bases and couplers allows a rich pallet of colors to be obtained.

The so-called “permanent” color obtained using these oxidation dyes must moreover satisfy a number of requirements. Thus, it should make it possible to obtain shades of the desired intensity and exhibit good resistance toward external agents (light, adverse weather conditions, washing, permanent waving, perspiration, rubbing).

The dyes should also make it possible to cover gray hair and should finally be the least selective possible, that is to say should make it possible to obtain the smallest possible differences in color right along the same keratinous fiber, which may indeed be differently sensitized (i.e. damaged) between its tip and its root.

Compositions for the oxidation dyeing of keratinous fibers containing, as oxidation dye precursors, certain substituted derivatives of para-phenylenediamine, have already been proposed, in particular in Patent Applications JP-11158046, JP-11158047 and JP-11158048. However, the colors obtained using these compositions are not always sufficiently intense, chromatic or resistant to the various attacks to which the hair may be subjected.

However, the applicant has now just discovered that it is possible to obtain novel dyes capable of leading to colors with a variety of shades, which are chromatic, intense, esthetic, not very selective and which exhibit good resistance to the various attacks to which the fibers may be subjected, by combining at least a first oxidation base chosen from certain para-phenylenediamine derivatives of formula (I) defined below and their addition salts with an acid and at least a second, suitably selected, oxidation base.

This discovery forms the basis of the present invention.

The first subject of the invention is therefore a composition for the oxidation dyeing of keratinous fibers, and in particular human keratinous fibers such as hair, characterized in that it comprises, in an appropriate dyeing medium:

at least a first oxidation base chosen from the substituted derivatives of para-phenylenediamine of the following formula (I), and their addition salts with an acid:

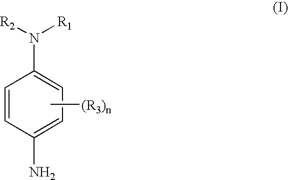

in which:

R₁ and R₂ can have one of the following meanings i) to v)

i) R₁ and R₂ simultaneously represent a radical —(CH₂)₂CHOHCH₂OH; or

ii) R₁ represents a radical —CH₂(CHOH)₄CH₂OH and R₂ represents a hydrogen atom, an alkyl or aryl radical or a heterocycle; or

iii) R₁ represents an alkyl or aryl radical or a heterocycle and R₂ represents an alkylene radical —(CH₂)ₘ— in which m is an integer equal to 2 or 3, said alkylene radical forming a ring together with the nitrogen atom, the carbon atom of the benzene ring carrying the nitrogen atom and one of the two carbon atoms of the benzene ring which are adjacent thereto, it being understood that when R₁ is an alkyl or aryl radical, then either R₁, or said alkylene radical is substituted with a radical containing at least one nitrogen, oxygen or sulfur atom;

iv) R₁ represents a radical —(CH₂CH₂O)ₚR₄ in which p is an integer between 2 and 8 inclusive, R₄ and R₂, which are identical or different, represent a hydrogen atom, an alkyl or aryl radical or a heterocycle;

v) R₁ and R₂ form, together with the nitrogen atom to which they are attached, a 5-, 6- or 7-membered saturated heterocycle, said heterocycle being substituted with at least one radical containing at least one carbon, nitrogen, oxygen or sulfur atom;

R₃ represents a halogen atom, an alkyl or aryl radical, a heterocycle, a heterocycle linked to the benzene ring of the formula (I) by an ether or thio bond, a cyano, nitro, hydroxyl, carboxyl, sulfo, alkoxy, aryloxy, cyanoamino, amino, anilino, ureido, sulfamylamino, mono- or dialkylsulfamylamino, alkylthio, arylthio, alkoxycarbonylamino, sulfonamido, carbamyl, mono- or dialkylcarbamylsulfamyl, sulfonyl, alkoxycarbonyl, azo, acyloxy, carbamyloxy, mono- or dialkylcarbamyloxy, silyl, silyloxy, aryloxycarbonylamino, imido, sulfinyl, phosphonyl, aryloxycarbonyl, acyl or mercapto radical;

said alkyl radicals comprising from 1 to 25 carbon atoms and being capable of being linear, branched or cyclic and of being substituted with one or more radicals and of then representing a mono- or polyhydroxyalkyl, alkoxyalkyl, aminoalkyl optionally substituted on the nitrogen atom, carboxyalkyl, alkylcarboxyalkyl, thioalkyl, alkylthioalkyl, cyanoalkyl, trifluoroalkyl, sulfoalkyl, phosphoalkyl or haloalkyl radical;

said alkoxy radicals comprising from 1 to 25 carbon atoms and being capable of being linear, branched or cyclic;

said aryl radicals comprising from 6 to 26 carbon atoms and being capable of being substituted with one or more radicals chosen from alkyl, substituted alkyl or alkoxy radicals;

the heterocycles being mono- or polycyclic, each ring comprising 3, 4, 5 or 6 members and being capable of containing one or more heteroatoms, it being understood that in the case of polycyclic heterocycles, at least one of the rings contains at least one heteroatom such as N, O or S;

n is an integer between 0 and 4; it being understood that when n is greater than 1, then the radicals R₃ may be identical or

different and may form with each other a 3-, 4-, 5- or 6-membered saturated or unsaturated ring;

with the proviso that:

1) when R₁ and R₂ have the meanings defined in point v), then the compounds of formula (I) do not contain more than 3 hydroxyl radicals;

2) when R₁ and R₂ have the meanings defined in point v) and R₁ and R₂ form a pyrrolidine ring substituted with a carbamoyl radical on the carbon at the alpha-position with respect to the nitrogen atom to which they are attached, then n is different from 0; or alternatively the pyrrolidine ring carries at least two substituents;

3) when R₁ and R₂ have the meanings defined in point v) and R₁ and R₂ form a pyrrolidine ring substituted with a hydroxymethyl radical on the carbon situated at the alpha-position with respect to the nitrogen atom to which they are attached, and n=0 or 1, then either said ring carries at least two additional substituents, or said ring comprises only a second substituent different from a hydroxyl radical on the carbon situated at the β-position with respect to the nitrogen atom and with respect to the carbon carrying said hydroxymethyl substituent; or alternatively when R₁ and R₂ have the meanings defined in point v) and R₁ and R₂ form a pyrrolidine ring substituted with a hydroxymethyl radical on the carbon situated at the alpha-position with respect to the nitrogen atom to which they are attached, and n=1, then R₃ is different from an alkyl, mono- or polyhydroxyalkyl radical;

4) when R₁ and R₂ have the meanings defined in point iii), the compounds of formula (I) must fulfill at least one of the following four conditions:

a) regardless of the value of n, the alkylene ring formed by the radical R₂ comprises a substituent in addition to the radical R₁; or

b) n is greater than 1; or

c) when n is equal to 1, then R₃ represents an aryl radical or a heterocycle; or

d) when n is equal to zero or to 1, then R₁ represents an aryl radical, a heterocycle or a substituted alkyl radical different from a monohydroxyalkyl radical;

5) when R₁ and R₂ have the meanings defined in point v), the groups R₁ and R₂ form a heterocycle different from piperazines and diazacycloheptanes.

and at least a second oxidation base chosen from heterocyclic oxidation bases, double bases, substituted para-aminophenols, ortho-aminophenols, para-phenylenediamine derivatives of the following formula (II), and their addition salts with an acid:

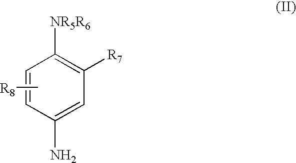

in which:

R₅ represents a hydrogen atom, a radical C₁-C₄ alkyl, C₁-C₄ monohydroxyalkyl, C₂-C₄ polyhydroxyalkyl, (C₁-C₄)alkoxy(C₁-C₄)alkyl, C₁-C₄ alkyl substituted with a nitrogen-containing group, a phenyl group or a 4′-aminophenyl group;

R₆ represents a hydrogen atom, a radical C₁-C₄ alkyl, C₁-C₄ monohydroxyalkyl, C₂-C₄ polyhydroxyalkyl, (C₁-C₄)alkoxy(C₁-C₄)alkyl, C₁-C₄ alkyl substituted with a nitrogen-containing group;

R₇ represents a hydrogen atom, a halogen atom such as a chlorine, bromine, iodine or fluorine atom, a C₁-C₄ alkyl, C₁-C₄ monohydroxyalkyl, C₁-C₄ hydroxyalkoxy, C₁-C₄ acetylaminoalkoxy, C₁-C₄ mesylaminoalkoxy or C₁-C₄ carbamoylaminoalkoxy radical,

R₈ represents a hydrogen or halogen atom or a C₁-C₄ alkyl radical;

it being understood that when R₅, R₆ and R₈ simultaneously represent a hydrogen atom, then R₇ does not designate a hydrogen atom, a chlorine atom or a methyl radical.

The dyeing composition in accordance with the invention leads to colors in a variety of shades, which are chromatic, intense, esthetic, which exhibit low selectivity and excellent properties of resistance to atmospheric agents such as light and adverse weather conditions and to perspiration and various treatments to which the hair may be subjected.

According to a particular embodiment, the oxidation base is chosen from the substituted derivatives of para-phenylenediamine of the following formula (I), and their addition salts with an acid:

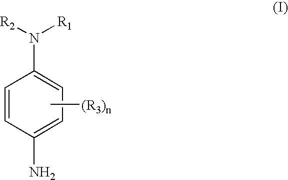

in which:

R₁ and R₂ can have one of the following meanings i) to v):

i) R₁ and R₂ simultaneously represent a radical —(CH₂)₂CHOHCH₂OH; or

ii) R₁ represents a radical —CH₂(CHOH)₄CH₂OH and R₂ represents a hydrogen atom, an alkyl radical; or

iv) R₁ represents a radical —(CH₂CH₂O)ₚR₄ in which p is an integer between 2 and 8 inclusive, R₄ and R₂, which are identical or different, represent a hydrogen atom, an alkyl radical;

v) R₁ and R₂ form, together with the nitrogen atom to which they are attached, a 5-, 6- or 7-membered saturated heterocycle, said heterocycle being substituted with at least one radical containing at least one carbon, nitrogen or oxygen atom, not situated at the meta-position with respect to the nitrogen atom of the heterocycle;

R₃ represents a halogen atom, an alkyl or aryl radical, or a heterocycle,

n is an integer equal to 0, 1 or 2;

According to one embodiment of the invention, the groups R1 and R2 form a heterocycle comprising a single heteroatom, nitrogen, for example a pyrrolidine heterocycle.

Among the substituted derivatives of para-phenylenediamine of formula (I) above, there may be mentioned most particularly 1-N,N-bis(3′,4′-dihydroxybutyl)-para-phenylenediamine, 1-N,N-bis(3′,4′-dihydroxybutyl)-3-methyl-para-phenylenediamine, 1-N,N-bis(3′,4′-dihydroxybutyl)-3-ethyl-para-phenylenediamine, 1-N,N-bis(3′,4′-dihydroxybutyl)-3-propyl-para-phenylenediamine, 1-N,N-bis(3′,4′-dihydroxybutyl)-3-methoxy-para-phenylenediamine, 1-N,N-bis(3′,4′-dihydroxybutyl)-3-ethoxy-para-phenylenediamine, 1-N,N-bis(3′,4′-dihydroxybutyl)-3-propyloxy-para-phenylenediamine, 1-N,N-bis(3′,4′-dihydroxybutyl)-3-hexyloxy-para-phenylenediamine, 1-N,N-bis(3′,4′-dihydroxybutyl)-3-(1″-N-3″,5″-dimethylpyrazolyl-para-phenylenediamine, 1-N,N-bis(3′,4′-dihydroxybutyl)-3-ureido-para-phenylenediamine, 1-N,N-bis(3′,4′-dihydroxybutyl)-3-trimethyl-1″,3″,3″-ureido-para-phenylenediamine, 1-N,N-bis(3′,4′-dihydroxybutyl)-3-dimethylamino-para-phenylenediamine, 1-N,N-bis(3′,4′-dihydroxybutyl)-3-methylthio-para-phenylenediamine, 1-N,N-bis(3′,4′-dihydroxybutyl)-3-ethylthio-para-phenylenediamine, 1-N,N-bis(3′,4′-dihydroxybutyl)-3-mercapto-para-phenylenediamine, 1-N,N-bis(3′,4′-dihydroxybutyl)-3-n-butylthio-para-phenylenediamine, 1-N,N-bis(3′,4′-dihydroxybutyl)-3-n-octylthio-para-phenylenediamine, 1-N,N-bis(3′,4′-dihydroxybutyl)-3-mercaptoethyl-para-phenylenediamine, 1-N,N-bis(3′,4′-dihydroxybutyl)-3-mercaptoethylthio-para-phenylenediamine, 1-N,N-bis(3′,4′-dihydroxybutyl)-3-hydroxyethylthio-para-phenylenediamine, 1-N-(2′,3′,4′,5′,6′-pentahydroxyhexyl)-para-phenylenediamine, 1-N-(2′,3′,4′,5′,6′-pentahydroxyhexyl)-3-methyl-para-phenylenediamine, 1-N-(2′,3′,4′,5′,6′-pentahydroxyhexyl)-3-isopropyl-para-phenylenediamine, 1-N-(2′,3′,4′,5′,6′-pentahydroxyhexyl)-3-methoxy-para-phenylenediamine, 1-N-(2′,3′,4′,5′,6′-pentahydroxyhexyl)-1-N-(4″,-N″-methylpiperidyl)-3-ethoxy-para-phenylenediamine, 1-N-(2′,3′,4′,5′,6′-pentahydroxyhexyl)-3-isopropyloxy-para-phenylenediamine, 1-N-(2′,3′,4′,5′,6′-pentahydroxyhexyl)-3-dimethylamino-para-phenylenediamine, 1-N-(2′,3′,4′,5′,6′-pentahydroxyhexyl)-3-methylthio-para-phenylenediamine, 1-N-(2′,3′,4′,5′,6′-pentahydroxyhexyl)-3-mercapto-para-phenylenediamine, 1-N-(hexyl)-1-N-(2′,3′,4′,5′,6′-pentahydroxyhexyl)-3-isopropyl-para-phenylenediamine, 1-N-(methyl)-1-N-(2′,3′,4′,5′,6′-pentahydroxyhexyl)-3-isooctyloxy-para-phenylenediamine, 1-N-(methyl)-1-N-(2′,3′,4′,5′,6′-pentahydroxyhexyl)-3-isopropyloxy-para-phenylenediamine, 1-N-(methyl)-1-N-(2′,3′,4′,5′,6′-pentahydroxyhexyl)-3-methyl-para-phenylenediamine, 1-N-(methyl)-1-N-(2′,3′,4′,5′,6′-pentahydroxyhexyl)-3-ethyl-para-phenylenediamine, 1-N-(methyl)-1-N-(2′,3′,4′,5′,6′-pentahydroxyhexyl)-3-hydroxyethyloxy-para-phenylenediamine, 1-N-(methyl)-1-N-(2′,3′,4′,5′,6′-pentahydroxyhexyl)-3-mercaptoethyloxy-para-phenylenediamine, 1-N-(methyl)-1-N-(2′,3′,4′,5′,6′-pentahydroxyhexyl)-para-phenylenediamine, 1-N-(phenyl)-1-N-(2′,3′,4′,5′,6′-pentahydroxyhexyl)-3-ethyloxy-para-phenylenediamine, 1-N-(4″-N-methylpiperidyl)-1-N-(2′,3′,4′,5′,6′-pentahydroxyhexyl)-3-ethyloxy-para-phenylenediamine, 4-N-(methyl)-4-N-(2′,3′,4′,5′,6′-pentahydroxyhexyl)amino-7-amino-1-methylindole, 1-N-(hydroxyethyloxyethyl)-1-N-(2′,3′,4′,5′,6′-pentahydroxyhexyl)-3-ethyl-para-phenylenediamine, and their addition salts with an acid.

Preferably, the oxidation base is chosen from 1-N,N-bis(3′,4′-dihydroxybutyl)-para-phenylenediamine, 1-N,N-bis(3′,4′-dihydroxybutyl)-3-methyl-para-phenylenediamine, 1-N,N-bis(3′,4′-dihydroxybutyl)-3-ethyl-para-phenylenediamine, 1-N,N-bis(3′,4′-dihydroxybutyl)-3-propyl-para-phenylenediamine, 1-N-(2′,3′,4′,5′,6′-pentahydroxyhexyl)-para-phenylenediamine, 1-N-(2′,3′,4′,5′,6′-pentahydroxyhexyl)-3-methyl-para-phenylenediamine, 1-N-(2′,3′,4′,5′,6′-pentahydroxyhexyl)-3-isopropyl-para-phenylenediamine, 1-N-(hexyl)-1-N-(2′,3′,4′,5′,6′-pentahydroxyhexyl)-3-isopropyl-para-phenylenediamine, 1-N-(methyl)-1-N-(2′,3′,4′,5′,6′-pentahydroxyhexyl)-3-methyl-para-phenylenediamine, 1-N-(methyl)-1-N-(2′,3′,4′,5′,6′-pentahydroxyhexyl)-3-ethyl-para-phenylenediamine, 1-N-(methyl)-1-N-(2′,3′,4′,5′,6′-pentahydroxyhexyl)-para-phenylenediamine, 1-N-(hydroxyethyloxyethyl)-1-N-(2′,3′,4′,5′,6′-pentahydroxyhexyl)-3-ethyl-para-phenylenediamine, and their addition salts with an acid.

There may also be mentioned most particularly 1-N-(3′,4′-dihydroxybutyl)-5-aminoindoline, 1-(2′-hydroxyethyl)-2-methyl-5-aminoindoline, 1-methyl-2-hydroxymethyl-5-aminoindoline, 6-methyl-2-hydroxyethyl-5-aminoindoline, 2-hydroxyethyloxyethyl-5-aminoindoline, 2-hydroxyethyloxyethyloxyethyloxyethyl-5-aminoindoline, 2-hydroxyethyloxyethyloxyethyloxyethyl-oxyethyloxyethyl-6-isopropyl-5-aminoindoline, 2-hydroxyethyl-3-methyl-5-aminoindoline, 2-hydroxyethyloxyethyloxyethyl-5-aminoindoline, 1-carboxymethyl-2,3,3-trimethyl-5-aminoindoline, 1-methylsulfonamidoethyl-3-methyl-5-aminoindoline, 1-ureidoethyl-6-methoxy-5-aminoindoline, 1-(2′,3′,4′,5′,6′-pentahydroxyhexyl)-5-aminoindoline, 1-N-(2′-mercaptoethyl)-5-aminoindoline, dimethyl ester 6-amino-1-methyl-1,2,3,4-tetrahydrofuro-[2,3,h]quinoline 4-methyl ester of phosphoric acid, 6-amino-1,2,2-trimethyl-4-trimethylsilanyloxy-1,2,3,4-tetrahydroquinoline, 6-amino-1-hexyl-2,2,7-trimethyl-4-mercaptomethyl-1,2,3,4-tetrahydroquinoline, 6-amino-1-(3′,4′-dihydroxybutyl)-2,2,3-trimethyl-1,2,3,4-tetra-hydroquinoline, 6-amino-1-(ethoxyethoxyethoxyethoxy-3′,4′-dihydroxybutyl)-2,2,3,7-tetramethyl-1,2,3,4-tetrahydroquinoline, 6-amino-1-(hydroxy-ethyloxyethyloxyethyloxyethyl)-2,2,3-trimethyl-1,2,3,4-tetrahydroquinoline, 6-amino-1-(hydroxyethyloxyethyl)-2,2,3-trimethyl-1,2,3,4-tetrahydroquinoline, 6-amino-1-(ethylbis(hydroxyethyloxyethyloxyethyloxyethyl))-2,2,3,7-tetramethyl-1,2,3,4-tetrahydroquinoline, 1-(carboxymethyl)-2,2,3,7-tetramethyl-1,2,3,4-tetrahydroquinoline, 1-(hydroxypropyl)-2,2,3-trimethyl-7-methoxy-1,2,3,4-tetrahydroquinoline, 6-amino-1-(hydroxyethyl-oxyethyloxyethyl)-2,2,3-trimethyl-7-isopropyl-1,2,3,4-tetrahydroquinoline, 6-amino-1-(hydroxyethyl-oxyethyloxyethyloxyethyloxyethyloxyethyloxyethyloxyethyl)-2,2,3-trimethyl-7-isopropyl-1,2,3,4-tetrahydroquinoline, 6-amino-1-(hydroxyethyloxyethyloxyethyloxy-ethyloxyethyloxyethyloxyethyloxyethyl)-2,2,3-trimethyl-1,2,3,4-tetrahydroquinoline, 6-amino-1-(mercaptoethyl)-1,2,3,4-tetrahydroquinoline, 6-amino-1-(3′,4′-dihydroxybutyl)-2,2,3-trimethyl-7-isopropyl-1,2,3,4-tetrahydroquinoline, 6-amino-1-(3′,4′-dihydroxybutyl)-2,2,7-trimethyl-4-hydroxymethyl-1,2,3,4-tetrahydroquinoline, 6-amino-1-(3′,4′-dihydroxybutyl)-2,2-dimethyl-4-hydroxymethyl-7-isopropyl-1,2,3,4-tetrahydroquinoline, 6-amino-1-(3-hydroxypropyl)-2,2-dimethyl-4-hydroxymethyl-7-isopropyl-1,2,3,4-tetrahydroquinoline, 6-amino-1-(hydroxyethyl-oxyethyloxyethyloxyethyloxyethyloxyethyloxyethyloxyethyl)-2,2-dimethyl-4-hydroxymethyl-7-isopropyl-1,2,3,4-tetrahydroquinoline, 6-amino-1-(hydroxyethyl-oxyethyloxyethyloxyethyl)-2,2-dimethyl-4-hydroxymethyl-7-isopropyl-1,2,3,4-tetrahydroquinoline, 6-amino-1-(hydroxyethyloxyethyloxyethyloxyethyloxyethyl)-2,2-dimethyl-7-isopropyl-1,2,3,4-tetrahydroquinoline, 6-amino-1-(hydroxyethyloxyethyloxyethyloxyethyloxyethyloxyethyl)-2,2-dimethyl-7-isopropyl-1,2,3,4-tetrahydroquinoline, 6-amino-1-(hydroxyethyloxyethyl)-2,2-dimethyl-1,2,3,4-tetrahydroquinoline, 6-amino-1,2,2,4,7-pentamethyl-3-hydroxy-1,2,3,4-tetrahydroquinoline, 6-amino-1-(3′-hydroxypropyl)-4-(hydroxy-ethyloxyethyloxyethyloxyethyl)-2,2-dimethyl-7-isopropyl-1,2,3,4-tetrahydroquinoline, 6-amino-1-(hydroxy-ethyloxyethyloxyethyloxyethyl)-4,4-dimethyl-1,2,3,4-tetrahydroquinoline, 6-amino-1-(3′,4′-dihydroxybutyl)-2,2-dimethyl-7-isopropyl-1,2,3,4-tetrahydroquinoline, 6-amino-1-(hydroxyethyloxyethyloxyethyloxyethyl)-4-(hydroxyethyloxyethyloxyethyloxyethyl)-2,2,7-trimethyl-1,2,3,4-tetrahydroquinoline, 6-amino-1-(hydroxyethyloxyethyloxyethyloxyethyl)-2,2-dimethyl-7-isopropyl-1,2,3,4-tetrahydroquinoline, 6-amino-1-(2′,3′,4′,5′,6′-pentahydroxyhexyl)-2,2,4-trimethyl-7-isopropyl-1,2,3,4-tetrahydroquinoline, 6-amino-1-(mercaptoethyl)-2,2,4-trimethyl-7-(2′,3′-dihydroxypropyloxy)-1,2,3,4-tetrahydro-quinoline, 6-amino-1-(3′,4′-dihydroxybutyl)-2,2,7-trimethyl-3-mercaptomethyl-1,2,3,4-tetrahydroquinoline, 6-amino-1-(ureidoethyl)-2,2,4-trimethyl-1,2,3,4-tetrahydroquinoline, 6-amino-2,2-dimethyl-7-chloro-1,2,3,4-tetrahydroquinoline-1-propylsulfonic acid, 6-amino-1-(4′-pyridinyl)-2,2,7-trimethyl-1,2,3,4-tetrahydroquinoline, 6-amino-1-(3′,4′-dihydroxybutyl)-2,2,4,7-tetramethyl-1,2,3,4-tetrahydroquinoline, 6-amino-1,7-diisopropyl-2,2-dimethyl-4-trimethylsilanyloxy-1,2,3,4-tetrahydroquinoline, 6-amino-1,2,2,4-tetramethyl-3-hydroxy-1,2,3,4-tetrahydroquinoline, 6-amino-1-bromo-2,2-dimethyl-4-mercapto-7-isopropyloxy-1,2,3,4-tetrahydroquinoline, and their addition salts with an acid.

There may also be mentioned most particularly 1-(4′-amino-3′-isopropyloxyphenyl)-2,6-dimethylpyrrolidine, 1-(4′-amino-3′-methylphenyl)-3-hydroxyethyloxypyrrolidine, 1-(4′-amino-3′-methylphenyl)-4-hydroxy-2-methylpyrrolidone, 1-(4′-amino-3′-methylphenyl)-3-methylsulfonamidopyrrolidine, 1-(4′-amino-3′-phenoxyphenyl)-3-methylsulfonamidopyrrolidine, 3-n-butylpyrrolidine-1-(4′-amino-3′-phenylsulfonic) acid, 1-(4′-amino-3′-acetylaminophenyl)-3-hydroxymethylpyrrolidine, 7-amino-4-(2′-methyl)-pyrrolydinylbenzofuran, 1-(4′-aminophenyl)-(2-(4″-aminophenoxymethyl)piperidine, 1-(4′-amino-3′-acetylphenyl)-4-hydroxypiperidine, 1-(4′-aminophenyl)-2-(hydroxyethyl)piperidine, 1-(4′-amino-3′-methoxyphenyl)-2,6-dihydroxymethylpiperidine, 1-(4′-amino-3′-isopropyloxyphenyl)-2,6-dimethylpiperidine, 1-(4′-amino-3′-isopropylphenyl)-2-hydroxymethylpiperidine, 1-(4′-amino-3′-isopropyloxyphenyl)-2-hydroxymethylpiperidine, 1-(4′-amino-3′-aminophenyl)-2-hydroxymethylpiperidine, 1-(4′-amino-3′-dimethylaminophenyl)-2-mercaptoethyloxyethylpiperidine, 1-(4′-amino-3′-(2′″,4′″-dichloro)anilinophenyl)-4-methylpiperidine, 1-(4′-aminophenyl)-4-methylpiperidine, 1-(4′-aminophenyl)-2,7-dimethylazacycloheptane, 1-(4′-amino-3′-methylphenyl)-2-methylazacycloheptane, 1-(4′-amino-3′-ureidophenyl)-3-hydroxyazacycloheptane, 1-(4′-amino-3′-sulfamoylaminophenyl)-2,7-dimethylazacycloheptane, 1-(4′-amino-3′-methylthiophenyl)-2,7-dimethylazacycloheptane, 1-N-4′-hydroxybutyl-1-N-(hydroxyethyloxyethyloxyethyl)-3-isopropyl-para-phenylenediamine, 1-N-methyl-1-N-(hydroxyethyloxyethyloxyethyl)-para-phenylenediamine, 1-N-phenyl-1-N-(hydroxyethyloxyethyl)-para-phenylenediamine, 1-N-benzyl-1-N-(hydroxyethyloxyethyloxyethyloxyethyloxyethyl)-3-trimethylsilyl-para-phenylenediamine, 1-N-methyl-1-N-(hydroxy-ethyloxyethyloxyethyloxyethyloxyethyloxyethyloxyethyloxyethyl)-3-trimethylsilyloxy-para-phenylenediamine, 1-N-ethyl-1-N-(methoxyethyloxyethyloxyethyloxyethyloxyethyl)-3-phenoxycarbonylamino-para-phenylenediamine, 1-N-methyl-1-N-(methoxyethyloxyethyloxyethyl)-3-(2′,5′-dioxopyrrolidinyl)-para-phenylenediamine, 1-N-ethyl-1-N-(hydroxyethyloxyethyloxyethyl)-3,4′-pyridinylthio-para-phenylenediamine, 1-N-propyl-1-N-(hydroxyethyloxyethyloxyethyl)-3-sulfinyl-para-phenylenediamine, 1-N-methyl-1-N-(hydroxyethyloxyethyl)-3-phenoxycarbonyl-para-phenylenediamine, 1-N,N-bis(hydroxyethyloxyethyloxyethyloxyethyl)-para-phenylenediamine, 1-N,N-bis(methoxyethyloxyethyloxyethyloxyethyl)-3-isopropyloxy-para-phenylenediamine, 1-N,N-bis(hydroxyethyloxyethyloxyethyl)-3-isopropyloxy-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyl)-3-isopropyl-para-phenylenediamine, 1-N,N-bis-(methoxyethyloxyethyloxyethyloxyethyloxyethyloxyethyloxyethyl)-3-methoxy-para-phenylenediamine, 1-N,N-bis-(hydroxyethyloxyethyloxyethyloxyethyloxyethyloxyethyloxyethyloxyethyl)-3-methyl-para-phenylenediamine, 1-N,N-bis(hydroxyethyloxyethyloxyethyloxyethyl)-3-isopropyloxy-para-phenylenediamine, 1-N,N-bis(hydroxyethyloxyethyl)-3-mercaptoethyl-para-phenylenediamine, 1-N,N-bis(benzyloxyethyloxyethyloxyethyl)-3-isopropyl-para-phenylenediamine, and their addition salts with an acid.

Preferably, the oxidation base is chosen from 1-(4′-amino-3′-methylphenyl)-3-hydroxyethyloxypyrrolidine, 1-(4′-amino-3′-methylphenyl)-4-hydroxy-2-methylpyrrolidine, 1-(4′-amino-3′-methylphenyl)-3-methylsulfonamidopyrrolidine, 1-(4′-amino-3′-phenoxyphenyl)-3-methylsulfonamidopyrrolidine, 1-(4′-aminophenyl)-2-(4″-aminophenoxymethyl)piperidine, 1-(4′-aminophenyl)-2-(hydroxyethyl)piperidine, 1-(4′-amino-3′-isopropylphenyl)-2-hydroxymethylpiperidine, 1-(4′-aminophenyl)-4-methylpiperidine, 1-(4′-aminophenyl)-2,7-dimethylazacycloheptane, 1-(4′-amino-3′-methylphenyl)-2-methylazacycloheptane, 1-(4′-amino-3′-ureidophenyl)-3-hydroxyazacycloheptane, 1-N-4′-hydroxybutyl-1-N-(hydroxyethyloxyethyloxyethyl)-3-isopropyl-para-phenylenediamine, 1-N-methyl-1-N-(hydroxyethyloxyethyloxyethyl)-para-phenylenediamine, 1-N,N-bis(hydroxyethyloxyethyloxyethyloxyethyl)-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyloxyethyloxyethyloxyethyloxyethyl)-3-methyl-para-phenylenediamine, 1-N,N-bis(benzyloxyethyloxyethyloxyethyl)-3-isopropyl-para-phenylenediamine, and their addition salts with an acid.

The para-phenylenediamine derivative(s) of formula (I) used as oxidation base in the dyeing composition in accordance with the invention preferably represent from 0.0001 to 20% by weight approximately, more preferably from 0.001 to 15% by weight and more preferably still from 0.01 to 10% by weight relative to the total weight of the composition.

Among the para-phenylenediamines of formula (II) which can be used as second oxidation base in the dyeing composition in accordance with the invention, there may be mentioned more particularly 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl, β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, and their addition salts with an acid.

Among the para-phenylenediamines of formula (II) above, there are most particularly preferred 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-β-acetylaminoethyloxy-para-phenylenediamine, and their addition salts with an acid.

More preferably still, there are preferred among the para-phenylenediamines of formula (II) above, 2-β-hydroxyethyl-para-phenylenediamine, N,N-bis(β-hydroxyethyl)-para-phenylenediamine, and their addition salts with an acid.

According to the invention, “double bases” is understood to mean the compounds containing at least two aromatic rings on which amino and/or hydroxyl groups are carried.

Among the double bases which can be used as second oxidation bases in the dyeing compositions in accordance with the invention, there may be mentioned in particular the compounds corresponding to the following formula (III), and their addition salts with an acid:

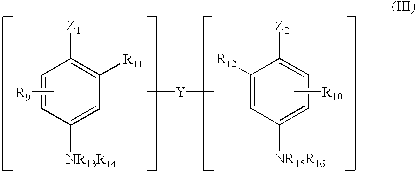

in which:

Z₁ and Z₂, which are identical or different, represent a hydroxyl or —NH₂ radical which may be substituted with a C₁-C₄ alkyl radical or with a linking arm Y;

the linking arm Y represents a linear or branched alkylene chain comprising from 1 to 14 carbon atoms, which may be interrupted by or which may end with one or more nitrogen-containing groups and/or one or more heteroatoms such as oxygen, sulfur or nitrogen atoms, and optionally substituted with one or more hydroxyl or C₁-C₆ alkoxy radicals;

R₉ and R₁₀ represent a hydrogen or halogen atom, a C₁-C₄ alkyl radical, a monohydroxy(C₁-C₄ alkyl) radical, a polyhydroxy(C₂-C₄ alkyl) radical, an amino(C₁-C₄ alkyl) radical or a linking arm Y;

R₁₁, R₁₂, R₁₃, R₁₄, R₁₅ and R₁₆, which are identical or different, represent a hydrogen atom, a linking arm Y or a C₁-C₄ alkyl radical;

it being understood that the compounds of formula (III) contain only one linking arm Y per molecule.

Among the nitrogen-containing groups of formula (III) above, there may be mentioned in particular the amino, mono(C₁-C₄)alkylamino, (C₁-C₄)dialkylamino, (C₁-C₄)trialkylamino, monohydroxy(C₁-C₄)alkylamino, imidazolinium and ammonium radicals.

Among the double bases of formula (III) above, there may be mentioned more particularly N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)-tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,5-dioxaoctane, and their addition salts with an acid.

Among these double bases of formula (III), N,N′-bis(β- hydroxyethyl)-N,N′-bis(4′-aminophenyl)-1,3-diaminopropanol, 1,8-bis(2,5-diaminophenoxy)-3,5-dioxaoctane or one of their addition salts with an acid are particularly preferred.

Among the substituted para-aminophenols which can be used as oxidation bases in the dyeing composition in accordance with the invention, there may be mentioned in particular the compounds of the following formula (IV), and their addition salts with an acid:

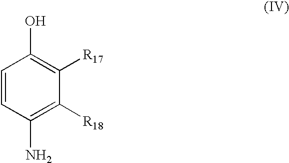

in which:

R₁₇ represents a hydrogen or halogen atom, a C₁-C₄ alkyl, monohydroxy (C₁-C₄ alkyl), (C₁-C₄) alkoxy (C₁-C₄) alkyl, amino (C₁-C₄ alkyl) or hydroxy(C₁-C₄)alkylamino(C₁-C₄ alkyl) radical,

R₁₈ represents a hydrogen or halogen atom, a C₁-C₄ alkyl, monohydroxy(C₁-C₄ alkyl), polyhydroxy(C₂-C₄ alkyl), amino(C₁-C₄ alkyl) , cyano(C₁-C₄ alkyl) or (C₁-C₄) alkoxy(C₁-C₄) alkyl radical, it being understood that at least one of the radicals R₁₇ or R₁₈ is different from a hydrogen atom.

Among the para-aminophenols of formula (IV) above, there may be mentioned more particularly para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, and their addition salts with an acid.

Among the ortho-aminophenols which can be used as oxidation bases in the dyeing compositions in accordance with the invention, there may be mentioned more particularly 2-aminophenol, 2-amino-5-methylphenol, 2-amino-6-methylphenol, 5-acetamido-2-aminophenol, and their addition salts with an acid.

Among the heterocyclic bases which can be used as oxidation bases in the dyeing compositions in accordance with the invention, there may be mentioned more particularly pyridine derivatives, pyrimidine derivatives, pyrazole derivatives, and their addition salts with an acid.

Among the pyridine derivatives, there may be mentioned more particularly the compounds described, for example, in Patents GB 1 026 978 and GB 1 153 196, such as 2,5-diaminopyridine, 2-(4-methoxyphenyl)amino-3-aminopyridine, 2,3-diamino-6-methoxypyridine, 2-(β-methoxyethyl)amino-3-amino-6-methoxypyridine, 3,4-diaminopyridine, and their addition salts with an acid.

Among the pyrimidine derivatives, there may be mentioned more particularly the compounds described, for example, in Patents DE 2 359 399; JP 88-169 571; JP 05 163 124; EP 0 770 375 or Patent Application WO 96/15765, such as 2,4,5,6-tetraaminopyrimidine, 4-hydroxy-2,5,6-triaminopyrimidine, 2-hydroxy-4,5,6-triaminopyrimidine, 6-hydroxy-2,4,5-triaminopyrimidine, 2,4-dihydroxy-5,6-diaminopyrimidine, 2,5,6-triaminopyrimidine, and the pyrazolopyrimidine derivatives such as those mentioned in Patent Application FR-A-2 750 048 and among which there may be mentioned pyrazolo[1,5-a]pyrimidine-3,7-diamine; 2,5-dimethylpyrazolo[1,5-a]pyrimidine-3,7-diamine; pyrazolo[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo-[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)-(2-hydroxyethyl)amino]ethanol, 2-[(7-aminopyrazolo[1,5-a]pyrimidin-3-yl)-(2-hydroxyethyl)amino]ethanol, 5,6-dimethylpyrazolo[1,5-a]pyrimidine-3,7-diamine, 2,6-dimethyl-pyrazolo[1,5-a]pyrimidine-3,7-diamine, 2,5,N7,N7-tetramethyl-pyrazolo[1,5-a]pyrimidine-3,7-diamine, 3-amino-5-methyl-7-imidazolylpropylaminopyrazolo[1,5-a]pyrimidine, their addition salts with an acid, and their tautomeric forms, when a tautomeric equilibrium exists.

Among the pyrazole derivatives, there may be mentioned more particularly the compounds described in Patents DE 3 843 892, DE 4 133 957 and Patent Applications WO 94/08969, WO 94/08970, FR-A-2 733 749 and DE 195 43 988 such as 4,5-diamino-1-methylpyrazole, 3,4-diaminopyrazole, 4,5-diamino-1-(4′-chlorobenzyl)pyrazole, 4,5-diamino-1,3-dimethylpyrazole, 4,5-diamino-1-(β-hydroxyethyl)pyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and their addition salts with an acid.

The oxidation base(s) used as second oxidation base preferably represent from 0.0005 to 12% by weight approximately of the total weight of the dyeing composition, and more preferably still from 0.005 to 6% by weight approximately of this weight.

According to a preferred embodiment of the invention, the dyeing composition contains at least one coupler.

These couplers may be chosen in particular from meta-phenylenediamines, meta-aminophenols, meta-diphenols and heterocyclic couplers such as, for example, indole derivatives, indoline derivatives, benzimidazole derivatives, benzomorpholine derivatives, sesamol derivatives, pyridine, pyrimidine and pyrazole derivatives, and their addition salts with an acid.

These couplers are more particularly chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)-propane, sesamol, 1-amino-2-methoxy-4,5-methylenedioxybenzene, α-naphthol, 2-methyl-1-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, and their addition salts with an acid.

When they are present, the coupler(s) preferably represent from 0.0001 to 15% by weight approximately relative to the total weight of the composition.

The dyeing composition in accordance with the invention may, in addition, contain one or more additional oxidation bases different from the oxidation bases described above and/or one or more direct dyes.

Among the additional oxidation bases which can be used according to the invention, there may be mentioned para-phenylenediamines other than those of formula (I) and (II) defined above, such as, for example, para-phenylenediamine, para-tolylenediamine, 2-chloro-para-phenylenediamine, and para-aminophenols other than those of formula (IV) defined above, such as 4-aminophenol, and their addition salts with an acid.

When they are present, the additional oxidation base(s) preferably represent from 0.0001 to 15% by weight approximately relative to the total weight of the composition.

In general, the addition salts with an acid which can be used in the context of the dyeing compositions of the invention are in particular chosen from hydrochlorides, hydrobromides, sulfates, tartrates, lactates and acetates.

The medium appropriate for dyeing (or carrier) generally consists of water or of a mixture of water and at least one organic solvent for solubilizing the compounds which might not be sufficiently soluble in water. By way of organic solvent, there may be mentioned for example lower C₁-C₄ alkanols such as ethanol and isopropanol; glycerol; glycols and glycol ethers such as 2-butoxyethanol, propylene glycol, monomethyl ether of propylene glycol, monoethyl ether and monomethyl ether of diethylene glycol, as well as aromatic alcohols such as benzyl alcohol or phenoxy-ethanol, similar products and mixtures thereof.

The solvents may be present in proportions preferably of between 1 and 40% by weight approximately relative to the total weight of the dyeing composition, and more preferably still between 5 and 30% by weight approximately.

The pH of the dyeing composition in accordance with the invention is generally between 3 and 12. It can be adjusted to the desired value by means of acidifying or alkalinizing agents normally used in dyeing keratinous fibers.

Among the acidifying agents, there may be mentioned, by way of example, inorganic or organic acids such as hydrochloric acid, orthophosphoric acid, carboxylic acids such as tartaric acid, citric acid, lactic acid and sulfonic acids.

Among the alkalinizing agents, there may be mentioned, by way of example, aqueous ammonia, alkali metal carbonates, alkanolamines such as mono-, di- and triethanolamines as well as their derivatives, oxyethylenated and/or oxypropylenated ethylenediamines and hydroxyalkylamines, sodium or potassium hydroxides and the compounds of the following formula (V):

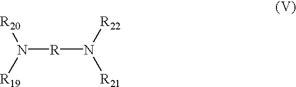

in which R is a propylene residue which is unsubstituted or substituted with a hydroxyl group or a C₁-C₄ alkyl radical; R₁₉, R₂₀, R₂₁ and R₂₂, which are identical or different, represent a hydrogen atom, a C₁-C₄ alkyl or C₁-C₄ hydroxyalkyl radical.

The acidifying agents are conventionally, by way of example, inorganic or organic acids such as hydrochloric acid, orthophosphoric acid, carboxylic acids such as tartaric acid, citric acid, lactic acid and sulfonic acids.

The dyeing composition in accordance with the invention may also contain various adjuvants conventionally used in hair-dyeing compositions, such as anionic, cationic, nonionic, amphoteric or zwitterionic surfactants or mixtures thereof, anionic, cationic, nonionic, amphoteric or zwitterionic polymers or mixtures thereof, inorganic or organic thickeners, reducing agents or antioxidants, penetrating agents, sequestering agents, perfumes, buffers, dispersing agents, conditioning agents such as for example silicones, film-forming agents, preservatives, opacifying agents, silicone or nonsilicone UV-screening agents, vitamins or provitamins.

The reducing agents or antioxidants may be chosen in particular from sodium sulfite, thioglycolic acid, thiolactic acid, sodium bisulfite, dehydroascorbic acid, hydroquinone, 2-methylhydroquinone, tert-butylhydroquinone and homogentisic acid, and they are in this case generally present in quantities which can vary between 0.05 and 1.5% by weight approximately relative to the total weight of the composition.

Preferably, the dyeing composition in accordance with the invention contains at least one nonionic surfactant in a proportion preferably varying between 0.1 and 20% by weight approximately relative to the total weight of the composition, and at least one cationic or amphoteric substantive polymer in a proportion preferably varying between 0.05 and 10% by weight relative to the total weight of the composition.

Preferably, the dyeing composition in accordance with the invention contains at least one thickening polymer comprising at least one hydrophilic unit and at least one fatty chain in a proportion preferably varying between 0.05 and 10% by weight relative to the total weight of the composition.

Of course, persons skilled in the art will be careful to choose the possible additional compound(s) mentioned above such that the advantageous properties intrinsically attached to the dyeing composition according to the invention are not, or not substantially, impaired by the addition(s) envisaged.

The dyeing composition in accordance with the invention may be provided in various forms, such as in the form of liquids, creams, gels, or in any other form appropriate for dyeing keratinous fibers, and in particular human hair.

Another subject of the invention is a method of oxidation dyeing keratinous fibers, and in particular human keratinous fibers such as hair, using the dyeing composition as defined above.

According to this method, at least one dyeing composition as defined above is applied to the fibers, the color being developed at acidic, neutral or alkaline pH with the aid of an oxidizing agent which is added just at the time of use to the dyeing composition or which is present in an oxidizing composition applied simultaneously or sequentially, separately.

According to a particularly preferred embodiment of the dyeing method according to the invention, the dyeing composition as defined above is mixed, at the time of use, with an oxidizing composition containing, in a medium appropriate for dyeing, at least one oxidizing agent present in a sufficient quantity to develop a color. The mixture obtained is then applied to the keratinous fibers and allowed to act for 3 to 50 minutes approximately, preferably 5 to 30 minutes approximately, after which they are rinsed, washed with shampoo, rinsed again and dried.

The oxidizing agent may be chosen from the oxidizing agents conventionally used for the oxidation dyeing of keratinous fibers, and among which there may be mentioned hydrogen peroxide, urea peroxide, alkali metal bromates, persalts such as perborates and persulfates, and oxidative enzymes such as peroxidases, laccases, tyrosinases and oxidoreductases among which there may be mentioned in particular pyranose oxidases, glucose oxidases, glycerol oxidases, lactate oxidases, pyruvate oxidases and uricases, said enzymes being optionally combined with their respective donors.

The pH of the oxidizing composition containing the oxidizing agent as defined above is such that after mixing with the dyeing composition, the pH of the resulting composition applied to the keratinous fibers preferably varies between 3 and 12 approximately, and more preferably still between 5 and 11. It is adjusted to the desired value by means of acidifying or alkalinizing agents normally used in dyeing keratinous fibers and as defined above.

The oxidizing composition as defined above may also contain various adjuvants conventionally used in hair-dyeing compositions and as defined above.

The composition which is finally applied to the keratinous fibers may be provided in various forms, such as in the form of liquids, creams, gels, or in any other form appropriate for dyeing keratinous fibers, and in particular human hair.

Another subject of the invention is a multicompartment device or multicompartment dyeing “kit” or any other multicompartment packaging system in which a first compartment contains the dyeing composition as defined above and a second compartment contains the oxidizing composition as defined above. These devices may be equipped with a means which makes it possible to deliver the desired mixture to the hair, such as the devices described in Patent FR-2 586 913 in the name of the applicant.

The following examples are intended to illustrate the invention.

### DYEING EXAMPLES 1 TO 5

The following dyeing compositions in accordance with the invention were prepared:

| EXAMPLES | 1 | 2 | 3 | 4 | 5 |
|---|---|---|---|---|---|
| 1-(4′-Amino-3′- | 2 × 10⁻³ | 2 × 10⁻³ | 2 × 10⁻³ | 2 × 10⁻³ | 2 × 10⁻³ |
| methylphenyl)-4-hydroxy-2- | mol | mol | mol | mol | mol |
| methylpyrrolidine | | | | | |
| dihydrochloride (substituted | | | | | |
| derivative of para- | | | | | |
| phenylenediamine of formula | | | | | |
| (I) in accordance with the | | | | | |
| invention) | | | | | |
| 2-Methyl-5-aminophenol | 3 × 10⁻³ | 3 × 10⁻³ | 3 × 10⁻³ | 3 × 10⁻³ | 3 × 10⁻³ |
| (coupler) | mol | mol | mol | mol | mol |
| 4-Amino-3-methylphenol | 10⁻³ | — | — | — | — |
| (second oxidation base) | mol | | | | |
| N,N-bis(β-hydroxyethyl)- | — | 10⁻³ | — | — | — |
| para-phenylenediamine | | mol | | | |
| sulfate (second oxidation | | | | | |
| base) | | | | | |
| N,N′-bis(β-hydroxyethyl)- | — | — | 10⁻³ | — | — |
| N,N′-bis(4-aminophenyl)-1,3- | | | mol | | |
| diaminopropanol tetrahydro- | | | | | |
| chloride (second oxidation | | | | | |
| base) | | | | | |
| 4,5-Diamino-1-methylpyrazole | — | — | — | 10⁻³ | — |
| dihydrochloride (second | | | | mol | |
| oxidation base) | | | | | |
| Ortho-aminophenol (second | — | — | — | — | 10⁻³ |
| oxidation base) | | | | | mol |
| Common dye carrier | (*) | (*) | (*) | (*) | (*) |
| Demineralized water qs | 100 g | 100 g | 100 g | 100 g | 100 g |

(*) Common Dye Carrier

| | |
|---|---|
| C₈—C₁₀ alkyl polyglucoside in aqueous solution | 5.4 g |
| at 60%, sold under the name ORAMIX CG 110 ® | |
| by the company SEPPIC | |
| Ethanol | 18.0 g |
| Benzyl alcohol | 1.8 g |
| Polyethylene glycol 400 | 2.7 g |
| Pentasodium salt of diethylenetriamine- | 1.08 g |
| pentaacetic acid in aqueous solution at | |
| 40%, sold under the name DISSOLUINE D-40 ® | |
| by the company AKZO | |
| Sodium metabisulfite | 0.205 g |
| Aqueous ammonia containing 20.5% of NH₃ | 10.0 g |

At the time of use, the dyeing compositions described above are mixed weight for weight with a solution of hydrogen peroxide at 20 volumes (6% by weight).

The mixtures thus prepared were applied for 30 minutes to locks of permanently waved natural gray hair which was 90% white. The locks were then rinsed, washed with a standard shampoo, rinsed again and then dried.

The hair was dyed in the following shades:

| Examples | Shades obtained |
|---|---|
| 1 | Strong red-purple |
| 2 | Strong purple |
| 3 | Strong purple |
| 4 | Strong red |
| 5 | Strong red-brown |

1. A composition for the oxidation dyeing of keratinous fibers, and in particular human keratinous fibers such as hair, characterized in that it comprises, in an appropriate dyeing medium:
at least a first oxidation base chosen from the substituted derivatives of para-phenylenediamine of the following formula (I), and their addition salts with an acid: 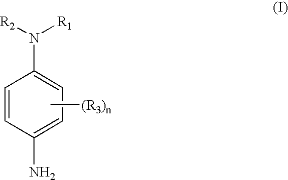
in which:
R₁ and R₂ can have one of the following meanings i) to v):
i) R₁ and R₂ simultaneously represent a radical —(CH₂)₂CHOHCH₂OH; or
ii) R₁ represents a radical —CH₂(CHOH)₄CH₂OH and R₂ represents a hydrogen atom, an alkyl or aryl radical or a heterocycle; or
iii) R₁ represents an alkyl or aryl radical or a heterocycle and R₂ represents an alkylene radical —(CH₂)ₘ— in which m is an integer equal to 2 or 3, said alkylene radical forming a ring together with the nitrogen atom, the carbon atom of the benzene ring carrying the nitrogen atom and one of the two carbon atoms of the benzene ring which are adjacent thereto, it being understood that when R₁ is an alkyl or aryl radical, then either R₁, or said alkylene radical is substituted with a radical containing at least one nitrogen, oxygen or sulfur atom;
iv) R₁ represents a radical —(CH₂CH₂O)ₚR₄ in which p is an integer between 2 and 8 inclusive, R₄ and R₂, which are identical or different, represent a hydrogen atom, an alkyl or aryl radical or a heterocycle;
v) R₁ and R₂ form, together with the nitrogen atom to which they are attached, a 5-, 6- or 7-membered saturated heterocycle, said heterocycle being substituted with at least one radical containing at least one carbon, nitrogen, oxygen or sulfur atom;
R₃ represents a halogen atom, an alkyl or aryl radical, a heterocycle, a heterocycle linked to the benzene ring of the formula (I) by an ether or thio bond, a cyano, nitro, hydroxyl, carboxyl, sulfo, alkoxy, aryloxy, cyanoamino, amino, anilino, ureido, sulfamylamino, mono- or dialkylsulfamylamino, alkylthio, arylthio, alkoxycarbonylamino, sulfonamido, carbamyl, mono- or dialkylcarbamylsulfamyl, sulfonyl, alkoxycarbonyl, azo, acyloxy, carbamyloxy, mono- or dialkylcarbamyloxy, silyl, silyloxy, aryloxycarbonylamino, imido, sulfinyl, phosphonyl, aryloxycarbonyl, acyl or mercapto radical;
said alkyl radicals comprising from 1 to 25 carbon atoms and being capable of being linear, branched or cyclic and of being substituted with one or more radicals and of then representing a mono- or polyhydroxyalkyl, alkoxyalkyl, aminoalkyl optionally substituted on the nitrogen atom, carboxyalkyl, alkylcarboxyalkyl, thioalkyl, alkylthioalkyl, cyanoalkyl, trifluoroalkyl, sulfoalkyl, phosphoalkyl or haloalkyl radical;
said alkoxy radicals comprising from 1 to 25 carbon atoms and being capable of being linear, branched or cyclic;
said aryl radicals comprising from 6 to 26 carbon atoms and being capable of being substituted with one or more radicals chosen from alkyl, substituted alkyl or alkoxy radicals;
the heterocycles being mono- or polycyclic, each ring comprising 3, 4, 5 or 6 members and being capable of containing one or more heteroatoms, it being understood that in the case of polycyclic heterocycles, at least one of the rings contains at least one heteroatom such as N, O or S;
n is an integer between 0 and 4; it being understood that when n is greater than 1, then the radicals R₃ may be identical or different and may form with each other a 3-, 4-, 5- or 6-membered saturated or unsaturated ring;
with the proviso that:
1) when R₁ and R₂ have the meanings defined in point v), then the compounds of formula (I) do not contain more than 3 hydroxyl radicals;
2) when R₁ and R₂ have the meanings defined in point v) and R₁ and R₂ form a pyrrolidine ring substituted with a carbamoyl radical on the carbon at the alpha-position with respect to the nitrogen atom to which they are attached, then n is different from 0; or alternatively the pyrrolidine ring carries at least two substituents;
3) when R₁ and R₂ have the meanings defined in point v) and R₁ and R₂ form a pyrrolidine ring substituted with a hydroxymethyl radical on the carbon situated at the alpha-position with respect to the nitrogen atom to which they are attached, and n=0 or 1, then either said ring carries at least two additional substituents, or said ring comprises only a second substituent different from a hydroxyl radical on the carbon situated at the β-position with respect to the nitrogen atom and with respect to the carbon carrying said hydroxymethyl substituent; or alternatively when R₁ and R₂ have the meanings defined in point v) and R₁ and R₂ form a pyrrolidine ring substituted with a hydroxymethyl radical on the carbon situated at the alpha-position with respect to the nitrogen atom to which they are attached, and n=1, then R₃ is different from an alkyl, mono- or polyhydroxyalkyl radical;
4) when R₁ and R₂ have the meanings defined in point iii), the compounds of formula (I) must fulfill at least one of the following four conditions:
a) regardless of the value of n, the alkylene ring formed by the radical R₂ comprises a substituent in addition to the radical R₁; or
b) n is greater than 1; or
c) when n is equal to 1, then R₃ represents an aryl radical or a heterocycle; or
d) when n is equal to zero or to 1, then R₁ represents an aryl radical, a heterocycle or a substituted alkyl radical different from a monohydroxyalkyl radical;
5) when R₁ and R₂ have the meanings defined in point v), the groups R₁ and R₂ form a heterocycle different from piperazines and diazacycloheptanes;
and at least a second oxidation base chosen from heterocyclic oxidation bases, double bases, substituted para-aminophenols, ortho-aminophenols, para-phenylenediamine derivatives of the following formula (II), and their addition salts with an acid: 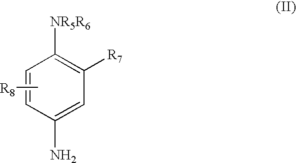
in which:
R₅ represents a hydrogen atom, a radical C₁-C₄ alkyl, C₁-C₄ monohydroxyalkyl, C₂-C₄ polyhydroxyalkyl, (C₁-C₄)alkoxy(C₁-C₄)alkyl, C₁-C₄ alkyl substituted with a nitrogen-containing group, a phenyl group or a 4′-aminophenyl group;
R₆ represents a hydrogen atom, a radical C₁-C₄ alkyl, C₁-C₄ monohydroxyalkyl, C₂-C₄ polyhydroxyalkyl, (C₁-C₄)alkoxy(C₁-C₄)alkyl, C₁-C₄ alkyl substituted with a nitrogen-containing group;
R₇ represents a hydrogen atom, a halogen atom such as a chlorine, bromine, iodine or fluorine atom, a C₁-C₄ alkyl, C₁-C₄ monohydroxyalkyl, C₁-C₄ hydroxyalkoxy, C₁-C₄ acetylaminoalkoxy, C₁-C₄ mesylaminoalkoxy or C₁-C₄ carbamoylaminoalkoxy radical,
R₈ represents a hydrogen or halogen atom or a C₁-C₄ alkyl radical;
it being understood that when R₅, R₆ and R₈ simultaneously represent a hydrogen atom, then R₇ does not designate a hydrogen atom, a chlorine atom or a methyl radical.

2. The composition as claimed in claim 1, in which the oxidation base is chosen from the substituted derivatives of para-phenylenediamine of the following formula (I), and their addition salts with an acid: 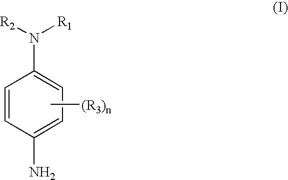
in which:
R₁ and R₂ can have one of the following meanings i) to v):
i) R₁ and R₂ simultaneously represent a radical —(CH₂)₂CHOHCH₂OH; or
ii) R₁ represents a radical —CH₂(CHOH)₄CH₂OH and R₂ represents a hydrogen atom, an alkyl radical; or
iv) R₁ represents a radical —(CH₂CH₂O)ₚR₄ in which p is an integer between 2 and 8 inclusive, R₄ and R₂, which are identical or different, represent a hydrogen atom, an alkyl radical;
v) R₁ and R₂ form, together with the nitrogen atom to which they are attached, a 5-, 6- or 7-membered saturated heterocycle, said heterocycle being substituted with at least one radical containing at least one carbon, nitrogen or oxygen atom, not situated at the meta-position with respect to the nitrogen atom of the heterocycle;
R₃ represents a halogen atom, an alkyl or aryl radical, or a heterocycle,
n is an integer equal to 0, 1 or 2.

3. The composition as claimed in claim 1 or 2, characterized in that the substituted derivatives of para-phenylenediamine of formula (I) are chosen from 1-N,N-bis(3′,4′-dihydroxybutyl)-para-phenylenediamine, 1-N,N-bis(3′,4′-dihydroxybutyl)-3-methyl-para-phenylenediamine, 1-N,N-bis(3′,4′-dihydroxybutyl)-3-ethyl-para-phenylenediamine, 1-N,N-bis(3′,4′-dihydroxybutyl)-3-propyl-para-phenylenediamine, 1-N,N-bis(3′,4′-dihydroxybutyl)-3-methoxy-para-phenylenediamine, 1-N,N-bis(3′,4′-dihydroxybutyl)-3-ethoxy-para-phenylenediamine, 1-N,N-bis(3′,4′-dihydroxybutyl)-3-propyloxy-para-phenylenediamine, 1-N,N-bis(3′,4′-dihydroxybutyl)-3-hexyloxy-para-phenylenediamine, 1-N,N-bis(3′,4′-dihydroxybutyl)-3-(1″-N-3″,5″-dimethylpyrazolyl-para-phenylenediamine, 1-N,N-bis(3′,4′-dihydroxybutyl)-3-ureido-para-phenylenediamine, 1-N,N-bis(3′,4′-dihydroxybutyl)-3-trimethyl-1″,3″,3″-ureido-para-phenylenediamine, 1-N,N-bis(3′,4′-dihydroxybutyl)-3-dimethylamino-para-phenylenediamine, 1-N,N-bis-(3′,4′-dihydroxybutyl)-3-methylthio-para-phenylenediamine, 1-N,N-bis(3′,4′-dihydroxybutyl)-3-ethylthio-para-phenylenediamine, 1-N,N-bis(3′,4′-dihydroxybutyl)-3-mercapto-para-phenylenediamine, 1-N,N-bis(3′,4′-dihydroxybutyl)-3-n-butylthio-para-phenylenediamine, 1-N,N-bis(3′,4′-dihydroxybutyl)-3-n-octylthio-para-phenylenediamine, 1-N,N-bis(3′,4′-dihydroxybutyl)-3-mercaptoethyl-para-phenylenediamine, 1-N,N-bis(3′,4′-dihydroxybutyl)-3-mercaptoethylthio-para-phenylenediamine, 1-N,N-bis(3′,4′-dihydroxybutyl)-3-hydroxyethylthio-para-phenylenediamine, 1-N-(2′,3′,4′,5′,6′-pentahydroxyhexyl)-para-phenylenediamine, 1-N-(2′,3′,4′,5′,6′-pentahydroxyhexyl)-3-methyl-para-phenylenediamine, 1-N-(2′,3′,4′,5′,6′-pentahydroxyhexyl)-3-isopropyl-para-phenylenediamine, 1-N-(2′,3′,4′,5′,6′-pentahydroxyhexyl)-3-methoxy-para-phenylenediamine, 1-N-(2′,3′,4′,5′,6′-pentahydroxy-hexyl)-1-N-(4″,-N″-methylpiperidyl)-3-ethoxy-para-phenylenediamine, 1-N-(2′,3′,4′,5′,6′-pentahydroxyhexyl)-3-isopropyloxy-para-phenylenediamine, 1-N-(2′,3′,4′,5′,6′-pentahydroxyhexyl)-3-dimethylamino-para-phenylenediamine, 1-N-(2′,3′,4′,5′,6′-pentahydroxyhexyl)-3-methylthio-para-phenylenediamine, 1-N-(2′,3′,4′,5′,6′-pentahydroxyhexyl)-3-mercapto-para-phenylenediamine, 1-N-(hexyl)-1-N-(2′,3′,4′,5′,6′-pentahydroxyhexyl)-3-isopropyl-para-phenylenediamine, 1-N-(methyl)-1-N-(2′,3′,4′,5′,6′-pentahydroxyhexyl)-3-isooctyloxy-para-phenylenediamine, 1-N-(methyl)-1-N-(2′,3′,4′,5′,6′-pentahydroxyhexyl)-3-isopropyloxy-para-phenylenediamine, 1-N-(methyl)-1-N-(2′,3′,4′,5′,6′-pentahydroxyhexyl)-3-methyl-para-phenylenediamine, 1-N-(methyl)-1-N-(2′,3′,4′,5′,6′-pentahydroxyhexyl)-3-ethyl-para-phenylenediamine, 1-N-(methyl)-1-N-(2′,3′,4′,5′,6′-pentahydroxyhexyl)-3-hydroxyethyloxy-para-phenylenediamine, 1-N-(methyl)-1-N-(2′,3′,4′,5′,6′-pentahydroxyhexyl)-3-mercaptoethyloxy-para-phenylenediamine, 1-N-(methyl)-1-N-(2′,3′,4′,5′,6′-pentahydroxyhexyl)-para-phenylenediamine, 1-N-(phenyl)-1-N-(2′,3′,4′,5′,6′-pentahydroxyhexyl)-3-ethyloxy-para-phenylenediamine, 1-N-(4″-N-methylpiperidyl)-1-N-(2′,3′,4′,5′,6′-pentahydroxyhexyl)-3-ethyloxy-para-phenylenediamine, 4-N-(methyl)-4-N-(2′,3′,4′,5′,6′-pentahydroxyhexyl)amino-7-amino-1-methylindole, 1-N-(hydroxyethyloxyethyl)-1-N-(2′,3′,4′,5′,6′-pentahydroxyhexyl)-3-ethyl-para-phenylenediamine, 1-N-(3′,4′-dihydroxybutyl)-5-aminoindoline, 1-(2′-hydroxyethyl)-2-methyl-5-aminoindoline, 1-methyl-2-hydroxymethyl-5-aminoindoline, 6-methyl-2-hydroxyethyl-5-aminoindoline, 2-hydroxyethyloxyethyl-5-aminoindoline, 2-hydroxyethyloxyethyloxyethyloxyethyl-5-aminoindoline, 2-hydroxyethyloxyethyloxyethyloxyethyloxyethyloxyethyl-6-isopropyl-5-aminoindoline, 2-hydroxyethyl-3-methyl-5-aminoindoline, 2-hydroxyethyloxyethyloxyethyl-5-aminoindoline, 1-carboxymethyl-2,3,3-trimethyl-5-aminoindoline, 1-methyl-sulfonamidoethyl-3-methyl-5-aminoindoline, 1-ureidoethyl-6-methoxy-5-aminoindoline, 1-(2′,3′,4′,5′,6′-pentahydroxyhexyl)-5-aminoindoline, 1-N-(2′-mercaptoethyl)-5-aminoindoline, dimethyl ester 6-amino-1-methyl-1,2,3,4-tetrahydrofuro-[2,3,h]quinoline 4-methyl ester of phosphoric acid, 6-amino-1,2,2-trimethyl-4-trimethylsilanyloxy-1,2,3,4-tetrahydroquinoline, 6-amino-1-hexyl-2,2,7-trimethyl-4-mercaptomethyl-1,2,3,4-tetrahydroquinoline, 6-amino-1-(3′,4′-dihydroxybutyl)-2,2,3-trimethyl-1,2,3,4-tetrahydroquinoline, 6-amino-1-(ethoxyethoxyethoxyethoxy-3′,4′-dihydroxybutyl)-2,2,3,7-tetramethyl-1,2,3,4-tetrahydroquinoline, 6-amino-1-(hydroxyethyloxyethyloxyethyloxyethyl)-2,2,3-trimethyl-1,2,3,4-tetrahydroquinoline, 6-amino-1-(hydroxyethyloxyethyl)-2,2,3-trimethyl-1,2,3,4-tetrahydroquinoline, 6-amino-1-(ethylbis(hydroxyethyloxyethyloxyethyloxyethyl))-2,2,3,7-tetramethyl-1,2,3,4-tetrahydroquinoline, 1-(carboxymethyl)-2,2,3,7-tetramethyl-1,2,3,4-tetrahydroquinoline, 1-(hydroxypropyl)-2,2,3-trimethyl-7-methoxy-1,2,3,4-tetrahydroquinoline, 6-amino-1-(hydroxyethyloxyethyloxyethyl)-2,2,3-trimethyl-7-isopropyl-1,2,3,4-tetrahydroquinoline, 6-amino-1-(hydroxyethyloxyethyloxyethyloxyethyloxyethyloxyethyloxyethyloxyethyl)-2,2,3-trimethyl-7-isopropyl-1,2,3,4-tetrahydroquinoline, 6-amino-1-(hydroxyethyloxyethyloxyethyloxyethyloxyethyloxyethyloxyethyloxyethyl)-2,2,3-trimethyl-1,2,3,4-tetrahydroquinoline, 6-amino-1-(mercaptoethyl)-1,2,3,4-tetrahydroquinoline, 6-amino-1-(3′,4′-dihydroxybutyl)-2,2,3-trimethyl-7-isopropyl-1,2,3,4-tetrahydroquinoline, 6-amino-1-(3′,4′-dihydroxybutyl)-2,2,7-trimethyl-4-hydroxymethyl-1,2,3,4-tetrahydroquinoline, 6-amino-1-(3′,4′-dihydroxybutyl)-2,2-dimethyl-4-hydroxymethyl-7-isopropyl-1,2,3,4-tetrahydroquinoline, 6-amino-1-(3-hydroxypropyl)-2,2-dimethyl-4-hydroxymethyl-7-isopropyl-1,2,3,4-tetrahydroquinoline, 6-amino-1-(hydroxyethyloxyethyloxyethyloxyethyloxyethyloxyethyloxyethyloxyethyl)-2,2-dimethyl-4-hydroxymethyl-7-isopropyl-1,2,3,4-tetrahydroquinoline, 6-amino-1-(hydroxyethyloxyethyloxyethyloxyethyl)-2,2-dimethyl-4-hydroxymethyl-7-isopropyl-1,2,3,4-tetrahydroquinoline, 6-amino-1-(hydroxyethyloxyethyloxyethyloxyethyloxyethyl)-2,2-dimethyl-7-isopropyl-1,2,3,4-tetrahydroquinoline, 6-amino-1-(hydroxyethyloxyethyloxyethyloxyethyloxyethyloxyethyl)-2,2-dimethyl-7-isopropyl-1,2,3,4-tetrahydroquinoline, 6-amino-1-(hydroxyethyloxyethyl)-2,2-dimethyl-1,2,3,4-tetrahydroquinoline, 6-amino-1,2,2,4,7-pentamethyl-3-hydroxy-1,2,3,4-tetrahydroquinoline, 6-amino-1-(3′-hydroxypropyl)-4-(hydroxyethyloxyethyloxyethyloxyethyl)-2,2-dimethyl-7-isopropyl-1,2,3,4-tetrahydroquinoline, 6-amino-1-(hydroxyethyloxyethyloxyethyloxyethyl)-4,4-dimethyl-1,2,3,4-tetrahydroquinoline, 6-amino-1-(3′,4′-dihydroxybutyl)-2,2-dimethyl-7-isopropyl-1,2,3,4-tetrahydroquinoline, 6-amino-1-(hydroxyethyloxyethyloxyethyloxyethyl)-4-(hydroxyethyloxyethyloxyethyloxyethyl)-2,2,7-trimethyl-1,2,3,4-tetrahydroquinoline, 6-amino-1-(hydroxyethyloxyethyloxyethyloxyethyl)-2,2-dimethyl-7-isopropyl-1,2,3,4-tetrahydroquinoline, 6-amino-1-(2′,3′,4′,5′,6′-pentahydroxyhexyl)-2,2,4-trimethyl-7-isopropyl-1,2,3,4-tetrahydroquinoline, 6-amino-1-(mercaptoethyl)-2,2,4-trimethyl-7-(2′,3′-dihydroxypropyloxy)-1,2,3,4-tetrahydroquinoline, 6-amino-1-(3′,4′-dihydroxybutyl)-2,2,7-trimethyl-3-mercaptomethyl-1,2,3,4-tetrahydroquinoline, 6-amino-1-(ureidoethyl)-2,2,4-trimethyl-1,2,3,4-tetrahydroquinoline, 6-amino-2,2-dimethyl-7-chloro-1,2,3,4-tetrahydroquinoline-1-propylsulfonic acid, 6-amino-1-(4′-pyridinyl)-2,2,7-trimethyl-1,2,3,4-tetrahydroquinoline, 6-amino-1-(3′,4′-dihydroxybutyl)-2,2,4,7-tetramethyl-1,2,3,4-tetrahydroquinoline, 6-amino-1,7-diisopropyl-2,2-dimethyl-4-trimethylsilanyloxy-1,2,3,4-tetrahydroquinoline, 6-amino-1,2,2,4-tetramethyl-3-hydroxy-1,2,3,4-tetrahydroquinoline, 6-amino-1-bromo-2,2-dimethyl-4-mercapto-7-isopropyloxy-1,2,3,4-tetrahydroquinoline, 1-(4′-amino-3′-isopropyloxyphenyl)-2,6-dimethylpyrrolidine, 1-(4′-amino-3′-methylphenyl)-3-hydroxyethyloxypyrrolidine, 1-(4′-amino-3′-methylphenyl)-4-hydroxy-2-methylpyrrolidone, 1-(4′-amino-3′-methylphenyl)-3-methylsulfonamidopyrrolidine, 1-(4′-amino-3′-phenoxyphenyl)-3-methylsulfonamidopyrrolidine, 3-n-butylpyrrolidine-1-(4′-amino-3′-phenylsulfonic) acid, 1-(4′-amino-3′-acetylaminophenyl)-3-hydroxymethylpyrrolidine, 7-amino-4-(2′-methyl)-pyrrolydinylbenzofuran, 1-(4′-aminophenyl)-2-(4″-aminophenoxymethyl)piperidine, 1-(4′-amino-3′-acetylphenyl)-4-hydroxypiperidine, 1-(4′-aminophenyl)-2-(hydroxyethyl)piperidine, 1-(4′-amino-3′-methoxyphenyl)-2,6-dihydroxymethylpiperidine, 1-(4′-amino-3′-isopropyloxyphenyl)-2,6-dimethylpiperidine, 1-(4′-amino-3′-isopropyloxyphenyl)-2-hydroxymethylpiperidine, 1-(4′-amino-3′-isopropyloxyphenyl)-2-hydroxymethylpiperidine, 1-(4′-amino-3′-aminophenyl)-2-hydroxymethylpiperidine, 1-(4′-amino-3′-dimethylaminophenyl)-2-mercaptoethyloxyethylpiperidine, 1-(4′-amino-3′-(2′″,4′″-dichloro)anilinophenyl)-4-methylpiperidine, 1-(4′-aminophenyl)-4-methylpiperidine, 1-(4′-aminophenyl)-2,7-dimethylazacycloheptane, 1-(4′-amino-3′-methylphenyl)-2-methylazacycloheptane, 1-(4′-amino-3′-ureidophenyl)-3-hydroxyazacycloheptane, 1-(4′-amino-3′-sulfamoylaminophenyl)-2,7-dimethylazacycloheptane, 1-(4′-amino-3′-methylthiophenyl)-2,7-dimethylazacycloheptane, 1-N-4′-hydroxybutyl-1-N-(hydroxyethyloxyethyloxyethyl)-3-isopropyl-para-phenylenediamine, 1-N-methyl-1-N-(hydroxyethyloxyethyloxyethyl)-para-phenylenediamine, 1-N-phenyl-1-N-(hydroxyethyloxyethyl)-para-phenylenediamine, 1-N-benzyl-1-N-(hydroxyethyloxyethyloxyethyloxyethyloxyethyl)-3-trimethylsilyl-para-phenylenediamine, 1-N-methyl-1-N-(hydroxyethyloxyethyloxyethyloxyethyloxyethyloxyethyloxyethyloxyethyl)-3-trimethylsilyloxy-para-phenylenediamine, 1-N-ethyl-1-N-(methoxyethyloxyethyloxyethyloxyethyloxyethyl)-3-phenoxycarbonylamino-para-phenylenediamine, 1-N-methyl-1-N-(methoxyethyloxyethyloxyethyl)-3-(2′,5′-dioxopyrrolidinyl)-para-phenylenediamine, 1-N-ethyl-1-N-(hydroxyethyloxyethyloxyethyl)-3,4′-pyridinylthio-para-phenylenediamine, 1-N-propyl-1-N-(hydroxyethyloxyethyloxyethyl)-3-sulfinyl-para-phenylenediamine, 1-N-methyl-1-N-(hydroxyethyloxyethyl)-3-phenoxycarbonyl-para-phenylenediamine, 1-N,N-bis(hydroxyethyloxyethyloxyethyloxyethyl)-para-phenylenediamine, 1-N,N-bis(methoxyethyloxyethyloxyethyloxyethyl)-3-isopropyloxy-para-phenylenediamine, 1-N,N-bis-(hydroxyethyloxyethyloxyethyl)-3-isopropyloxy-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyl)-3-isopropyl-para-phenylenediamine, 1-N,N-bis-(methoxyethyloxyethyloxyethyloxyethyloxyethyloxyethyloxyethyl)-3-methoxy-para-phenylenediamine, 1-N,N-bis-(hydroxyethyloxyethyloxyethyloxyethyloxyethyloxyethyloxyethyloxyethyl)-3-methyl-para-phenylenediamine, 1-N,N-bis(hydroxyethyloxyethyloxyethyloxyethyl)-3-isopropyloxy-para-phenylenediamine, 1-N,N-bis(hydroxyethyloxyethyl)-3-mercaptoethyl-para-phenylenediamine, 1-N,N-bis(benzyloxyethyloxyethyloxyethyl)-3-isopropyl-para-phenylenediamine, and their addition salts with an acid.

4. The composition as claimed in any one of the preceding claims, in which the oxidation base is chosen from 1-N,N-bis(3′,4′-dihydroxybutyl)-para-phenylenediamine, 1-N,N-bis(3′,4′-dihydroxybutyl)-3-methyl-para-phenylenediamine, 1-N,N-bis(3′,4′-dihydroxybutyl)-3-ethyl-para-phenylenediamine, 1-N,N-bis(3′,4′-dihydroxybutyl)-3-propyl-para-phenylenediamine, 1-N-(2′,3′,4′,5′,6′-pentahydroxyhexyl)-para-phenylenediamine, 1-N-(2′,3′,4′,5′,6′-pentahydroxyhexyl)-3-methyl-para-phenylenediamine, 1-N-(2′,3′,4′,5′,6′-pentahydroxyhexyl)-3-isopropyl-para-phenylenediamine, 1-N-(hexyl)-1-N-(2′,3′,4′,5′,6′-pentahydroxyhexyl)-3-isopropyl-para-phenylenediamine, 1-N-(methyl)-1-N-(2′,3′,4′,5′,6′-pentahydroxyhexyl)-3-methyl-para-phenylenediamine, 1-N-(methyl)-1-N-(2′,3′,4′,5′,6′-pentahydroxyhexyl)-3-ethyl-para-phenylenediamine, 1-N-(methyl)-1-N-(2′,3′,4′,5′,6′-pentahydroxyhexyl)-para-phenylenediamine, 1-N-(hydroxyethyloxyethyl)-1-N-(2′,3′,4′,5′,6′-pentahydroxyhexyl)-3-ethyl-para-phenylenediamine, and their addition salts with an acid.

5. The composition as claimed in any one of claims 1 to 3, in which the oxidation base is chosen from 1-(4′-amino-3′-methylphenyl)-3-hydroxyethyloxypyrrolidine, 1-(4′-amino-3′-methylphenyl)-4-hydroxy-2-methylpyrrolidine, 1-(4′-amino-3′-methylphenyl)-3-methylsulfonamidopyrrolidine, 1-(4′-amino-3′-phenoxyphenyl)-3-methylsulfonamidopyrrolidine, 1-(4′-amino-phenyl)-2-(4″-aminophenoxymethyl)piperidine, 1-(4′-aminophenyl)-2-(hydroxyethyl)piperidine, 1-(4′-amino-3′-isopropylphenyl)-2-hydroxymethylpiperidine, 1-(4′-aminophenyl)-4-methylpiperidine, 1-(4′-aminophenyl)-2,7-dimethylazacycloheptane, 1-(4′-amino-3′-methylphenyl)-2-methylazacycloheptane, 1-(4′-amino-3′-ureidophenyl)-3-hydroxyazacycloheptane, 1-N-4′-hydroxybutyl-1-N-(hydroxyethyloxyethyloxyethyl)-3-isopropyl-para-phenylenediamine, 1-N-methyl-1-N-(hydroxyethyloxyethyloxyethyl)-para-phenylenediamine, 1-N,N-bis(hydroxyethyloxyethyloxyethyloxyethyl)-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyl)-3-isopropyl-para-phenylenediamine, 1-N,N-bis(hydroxyethyloxyethyloxyethyloxyethyloxyethyloxyethyloxyethyloxyethyl)-3-methyl-para-phenylenediamine, 1-N,N-bis-(benzyloxyethyloxyethyloxyethyl)-3-isopropyl-para-phenylenediamine, and their addition salts with an acid.

6. The composition as claimed in any one of the preceding claims, characterized in that the para-phenylenediamine derivative(s) of formula (I) represent from 0.0001 to 20% by weight of the total weight of the composition.

7. The composition as claimed in claim 1 or 2, characterized in that the groups R1 and R2 form a pyrrolidine heterocycle.

8. The composition as claimed in any one of the preceding claims, characterized in that the para-phenylenediamines of formula (II) are chosen from 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N,N-(ethyl, β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, and their addition salts with an acid.

9. The composition as claimed in claim 8, characterized in that the para-phenylenediamines of formula (II) are chosen from 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-β-acetylaminoethyloxy-para-phenylenediamine, and their addition salts with an acid.

10. The composition as claimed in claim 9, characterized in that the para-phenylenediamines of formula (II) are chosen from 2-β-hydroxyethyl-para-phenylenediamine, N,N-bis(β- hydroxyethyl)-para-phenylenediamine, and their addition salts with an acid.

11. The composition as claimed in any one of claims 1 to 7, characterized in that the double bases are chosen from the compounds of the following formula (III), and their addition salts with an acid: 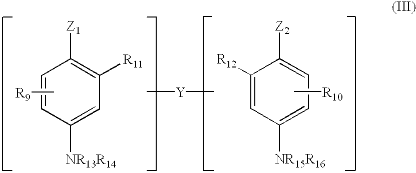
in which:
Z₁ and Z₂, which are identical or different, represent a hydroxyl or —NH₂ radical which may be substituted with a C₁-C₄ alkyl radical or with a linking arm Y;
the linking arm Y represents a linear or branched alkylene chain comprising from 1 to 14 carbon atoms, which may be interrupted by or which may end with one or more nitrogen-containing groups and/or one or more heteroatoms such as oxygen, sulfur or nitrogen atoms, and optionally substituted with one or more hydroxyl or C₁-C₆ alkoxy radicals;
R₉ and R₁₀ represent a hydrogen or halogen atom, a C₁-C₄ alkyl radical, a monohydroxy(C₁-C₄ alkyl) radical, a polyhydroxy(C₂-C₄ alkyl) radical, an amino(C₁-C₄ alkyl) radical or a linking arm Y;
R₁₁, R₁₂, R₁₃, R₁₄, R₁₅ and R₁₆, which are identical or different, represent a hydrogen atom, a linking arm Y or a C₁-C₄ alkyl radical;
it being understood that the compounds of formula (III) contain only one linking arm Y per molecule.

12. The composition as claimed in claim 11, characterized in that the double bases of formula (III) are chosen from N,N′-bis(β-hydroxyethyl)-N,N′-bis(4′-aminophenyl)-1,3-diaminopropanol, N,N′-bis(β- hydroxyethyl)-N,N′-bis-(4′-aminophenyl)ethylenediamine, N,N′-bis(4-aminophenyl)-tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine, 1,8-bis(2,5-diaminophenoxy)-3,5-dioxaoctane, and their addition salts with an acid.

13. The composition as claimed in any one of claims 1 to 7, characterized in that the substituted para-aminophenols are chosen from the compounds of the following formula (IV), and their addition salts with an acid: 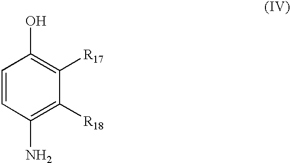
in which:
R₁₇ represents a hydrogen or halogen atom, a C₁-C₄ alkyl, monohydroxy(C₁-C₄ alkyl), (C₁-C₄)alkoxy(C₁-C₄)alkyl, amino(C₁-C₄ alkyl) or hydroxy(C₁-C₄)alkylamino(C₁-C₄ alkyl) radical,
R₁₈ represents a hydrogen or halogen atom, a C₁-C₄ alkyl, monohydroxy(C₁-C₄ alkyl), polyhydroxy(C₂-C₄ alkyl), amino(C₁-C₄ alkyl), cyano(C₁-C₄ alkyl) or (C₁-C₄)alkoxy(C₁-C₄)alkyl radical,
it being understood that at least one of the radicals R₁₇ or R₁₈ is different from a hydrogen atom.

14. The composition as claimed in claim 13, characterized in that the para-aminophenols of formula (IV) are chosen from para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol, 4-amino-2-fluorophenol, and their addition salts with an acid.

15. The composition as claimed in any one of claims 1 to 7, characterized in that the ortho-aminophenols are chosen from 2-aminophenol, 2-amino-5-methylphenol, 2-amino-6-methylphenol, 5-acetamido-2-aminophenol, and their addition salts with an acid.

16. The composition as claimed in any one of claims 1 to 7, characterized in that the heterocyclic bases are chosen from pyridine derivatives, pyrimidine derivatives, pyrazole derivatives, and their addition salts with an acid.

17. The composition as claimed in any one of the preceding claims, characterized in that the oxidation base(s) used as second oxidation base represent from 0.0005 to 12% by weight of the total weight of the dyeing composition.

18. The composition as claimed in claim 17, characterized in that the oxidation base(s) used as second oxidation base represent from 0.005 to 6% by weight of the total weight of the dyeing composition.

19. The composition as claimed in any one of the preceding claims, characterized in that it contains at least one coupler chosen from meta-phenylenediamines, meta-aminophenols, meta-diphenols and heterocyclic couplers, and their addition salts with an acid.

20. The composition as claimed in claim 19, characterized in that the couplers are chosen from 2-methyl-5-aminophenol, 5-N-(β-hydroxyethyl)amino-2-methylphenol, 3-aminophenol, 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)-propane, sesamol, 1-amino-2-methoxy-4,5-methylenedioxybenzene, α-naphthol, 2-methyl-1-naphthol, 6-hydroxyindole, 4-hydroxyindole, 4-hydroxy-N-methylindole, 6-hydroxyindoline, 2,6-dihydroxy-4-methylpyridine, 1-H-3-methylpyrazol-5-one, 1-phenyl-3-methylpyrazol-5-one, and their addition salts with an acid.

21. The composition as claimed in claim 19 or 20, characterized in that the coupler(s) represent from 0.0001 to 15% by weight relative to the total weight of the composition.

22. The composition as claimed in any one of the preceding claims, characterized in that it contains one or more additional oxidation bases chosen from para-phenylenediamine, para-tolylenediamine, 2-chloro-para-phenylenediamine, 4-aminophenol, and their addition salts with an acid.

23. The composition as claimed in any one of the preceding claims, characterized in that the addition salts with an acid are chosen from hydrochlorides, hydrobromides, sulfates, tartrates, lactates and acetates.

24. A method for the oxidation dyeing of keratinous fibers, and in particular human keratinous fibers such as hair, characterized in that at least one dyeing composition as defined in any one of claims 1 to 23 is applied to said fibers, the color being developed at acidic, neutral or alkaline pH with the aid of an oxidizing agent which is added just at the time of use to the dyeing composition or which is present in an oxidizing composition applied simultaneously or sequentially, separately.

25. The method as claimed in claim 24, characterized in that the oxidizing agent is chosen from hydrogen peroxide, urea peroxide, alkali metal bromates, persalts and oxidative enzymes.

26. A multicompartment device, or multicompartment dyeing “kit”, in which a first compartment contains a dyeing composition as defined in any one of claims 1 to 23 and a second compartment contains an oxidizing composition.
